(19) United States
(12) Patent Application Publication
(10) Pub. No.: US 2025/0081968 A1
(43) Pub. Date: Mar. 13, 2025

(54) Aqueous Hypochlorite Disinfectant Solution With Good Stability And Anti-Microbial Activity And Its Use

(71) Applicant: Fresenius Medical Care Deutschland GmbH, Bad Homburg (DE)

(72) Inventor: Misha Zou, Shanghai (CN)

(21) Appl. No.: 18/292,686

(22) PCT Filed: Jul. 26, 2022
(86) PCT No.: PCT/CN2022/107811

§ 371 Date: Jan. 26, 2024

(30) Foreign Application Priority Data
Jul. 29, 2021 (CN) 202110867697.2

(51) Int. Cl. A01N 59/00; A01N 25/22; A01P 1/00; A61L 2/18

(52) U.S. Cl. CPC A01N 59/00; A01N 25/22; A01P 1/00; A61L 2/18; A61L 2202/24

(57) ABSTRACT

The present disclosure relates generally to aqueous disinfectant solutions comprising hypochlorite which can be used for disinfection of medical devices, in particular hemodialysis machines. The aqueous disinfectant solutions have both a good stability and a high anti-microbial activity. The disclosure also relates to the use of said aqueous disinfectant solutions in disinfecting medical devices, in particular hemodialysis machines.

### CROSS-REFERENCE TO RELATED APPLICATIONS

The present application is the national stage entry of International Application No. PCT/CN2022/107811, filed on Jul. 26, 2022, and claims priority to Application No. CN 202110867697.2, filed in the People's Republic of China on Jul. 29, 2021, the disclosures of which are expressly incorporated herein in their entirety by reference thereto.

### TECHNICAL FIELD

The present disclosure relates generally to aqueous disinfectant solutions that can be used to disinfect medical devices, in particular hemodialysis machines, and more specifically to aqueous disinfectant solutions comprising hypochlorite, which have both good stability and anti-microbial activity.

The disclosure also relates to the use of said aqueous disinfectant solution in disinfecting medical devices, in particular hemodialysis machines.

### BACKGROUND

Hypochlorite bleach has been widely used in medical facilities in various environments due to its excellent spore killing effectiveness. Inorganic chlorine solutions are used as disinfectants for mannequins, laundries, dental appliances, hydrotherapy tanks, regulated medical waste prior to disposal, and water distribution systems in hemodialysis centers and hemodialysis machines.

Sodium hypochlorite has broad-spectrum antimicrobial activity, leaves no toxic residues, removes organic matter from dry and fixed surfaces, and has a low incidence of serious toxicity.

However, sodium hypochlorite is inherently unstable and will disproportionate to chlorates (e.g., NaClO2, NaClO3, etc.) and chlorides. When sodium hypochlorite is used as a concentrated aqueous solution, the high concentration of hypochlorite leads to high decomposition rates.

The shelf life of commercial disinfectants has become a major challenge due to poor stability. Typically, the shelf life of bleach (without stabilizers) of commercial disinfectants on the Chinese market is about 3-6 months. Some household or industrial disinfectants have a shelf life of about 3-6 months through the addition of bleach stabilizer can extend the shelf life up to 12 months.

CN 101679921 A discloses a stabilized concentrated cleaning solution comprising from about 1-40 wt % of hydroxide ion source, about 5-80 wt % of hypochlorite ion source, and about 0.0001-2.00 wt % of chelate ion source. Said chelating agent may be EDT, MGDA, NTA, IDS, polyacrylate or a combination thereof. In addition, the concentrated cleaning solution may contain any other ingredients used in conventional cleaning solutions, such as multivalent chelating agents, surfactants, preservatives, cleaning agents, solubilizers, solvents, etc.

U.S. Pat. No. 5,972,866 A discloses a method for cleaning a food manufacturing surface, said method comprising thickening The step of applying a non-corrosive composition to a surface, said composition comprising:
(a) about 0.1-10 weight percent of effective chlorine provided by hypochlorite bleach to reduce contamination on the surface;
(b) about 0.1-25 wt % the source of alkali metal hydroxide alkalinity which effectively provides a pH of about 10-14 for said composition;
(c) About 0.1-10 wt % inorganic thickener to promote said thickened non-corrosive composition in application to adhesion of the surface;
(d) about 0.05-5 wt % fatty acid stabilizer to maintain said bleaching agent, thickening a homogeneous mixture of the agent and the base source; and
(e) about 0.1-10 wt % of an anionic surfactant having effectively provide detergency to the thickened non-corrosive composition;
(f) an equilibrium amount of water;
   wherein said thickened non-Corrosive compositions have disinfectant and anti-microbial efficacy.

US 2006/0089285 A1 discloses a storage stable aqueous alkaline liquid containing a chlorine bleach body cleaning composition comprising: a chlorine bleach capable of forming hypochlorite in water; a bleach stabilizer, selected from having at least one NCl-, NHCl- or NCl₂-compound capable of reacting with said hypochlorite to form NH or NH₂ portion of the compound; and about 5-50% by weight of the metal hydroxide; said composition having a pH of at least 11.5. The cleaning compositions can be used to clean and disinfect hard surfaces for domestic or industrial use.

The disinfectant used for disinfection of hemodialysis machines is usually indicated as “special disinfectant for hemodialysis equipment”.

The manufacturer of the hemodialysis machine usually recommends the appropriate product after verifying and evaluating the safety and suitability of the disinfectant. For example, Fresenius Medical Care dialysis machines should use Sporotal (which contains sodium hypochlorite, potassium hydroxide, and potassium silicate) and Citrosteril (which contains citric acid monohydrate, lactic acid, and malic acid).

For disinfectants used in medical devices, the addition of any new ingredients should be evaluated very carefully to avoid the risk of unknown toxicity. More specifically for hemodialysis machines, the toxic agent components used for the disinfection have a certain possibility to enter the human body through the dialysis membrane, so the risk is higher, and more caution is needed with the introduction of new substances.

Thus, there is a real and unmet need in the field for chlorine-containing disinfectants that can be safely applied for the disinfection of medical devices, particularly hemodialysis machines, and have better stability while still providing excellent anti-microbial activity.

### SUMMARY

The present disclosure provides an aqueous disinfectant solution that can be used for the disinfection of medical devices, especially hemodialysis machines, with an improved stability while still providing excellent anti-microbial activity.

This present disclosure provides an aqueous disinfectant solution comprising 20-50 g/L of a hypochlorite, and 3.5-7.0 wt % of one or more metal hydroxides selected from an alkali metal or an alkaline earth metal hydroxide, and 0.001-4.0 wt % of one or more salts of a phosphate compound.

The present disclosure also provides an aqueous disinfectant solution comprising 30-40 g/L of a hypochlorite, and 2.0-3.0 wt % of one or more salts of a phosphate compound.

### DETAILED DESCRIPTION

Hypochlorites, especially sodium hypochlorite, are widely used for the disinfection of medical devices because of its many advantages (e.g., broad-spectrum antimicrobial activity, removal of surface-dried immobilized organisms and biofilms, and low incidence of severe toxicity without leaving toxic residues). However, sodium hypochlorite is inherently unstable, and when used as a concentrated aqueous solution, high hypochlorite concentrations result in high decomposition rates. The inventors of the patent application of the present disclosure found that by combining specific concentrations of hypochlorite, metal hydroxide and phosphate compounds it was possible to provide aqueous solutions of disinfectants with both higher stability (storage stability, long storage period) and anti-microbial activity, thus solving the problems of the prior art.

According to an aspect of the present disclosure, there is provided an aqueous disinfectant solution comprising: a disinfectant in the form of 20-50 g/L of hypochlorite and 3.5-7.0 wt % of alkali metal hydroxide or alkaline earth metal hydroxide, and of 0.001-4.0 wt % of salts of a phosphate compound.

In one embodiment, said hypochlorite is sodium hypochlorite or potassium hypochlorite, which is used in the disinfectant water. The concentration in the solution may be, for example, 20, 25, 28, 29, 30, 31, 32, 33, 35, 38, 40, 45 or 50 g/L, including a range between any two of the above point values.

In one embodiment, said hypochlorite 25˜45 g/L of aqueous solution, preferably 28˜38 g/L of aqueous solution, preferably 30˜35 g/L aqueous solution.

In the present disclosure, said alkali metals include sodium, potassium, lithium, etc.; said alkaline earth metals include calcium, magnesium, etc.

In one embodiment, said alkali metal or alkaline earth metal hydroxide is sodium hydroxide or potassium hydroxide.

In one embodiment, the content of said alkali metal or alkaline earth metal hydroxide is 4.0-6.0 wt %, preferably 4.0-5.0 wt %.

Said phosphoric acid compound in the scope of the present disclosure comprises orthophosphoric acid, pyrophosphoric acid, cyclic phosphoric acid, one or more polyphosphate and superphosphate. The salt of said phosphate compound includes an ammonium salt or a metal salt, in particular, an alkali metal salt, such as sodium salts, potassium salts and lithium salts, preferably potassium and sodium salts, more preferably potassium salts.

In one embodiment of the present disclosure, said salt of the phosphate compound is a polyphosphate alkali metal salt, preferably potassium polyphosphate and sodium polyphosphate, more preferably potassium tripolyphosphate (KTPP). In one embodiment, said phosphate compound content is 0.001-4.0 wt %, preferably 0.01-3.5 wt %, more preferably 0.1-3.0 wt %, most preferably 0.5-2.0 wt % or 0.5-1.0 wt %.

According to another aspect of the present disclosure, there is provided an aqueous disinfectant solution comprising: 30-40 g/L of hypochlorite, and 2.0-3.0 wt % of salt of phosphate compounds.

In one embodiment of this aspect, said hypochlorite is sodium hypochlorite or potassium hypochlorite. In one embodiment, said hypochlorite content is 30-40 g/L, preferably 33-35 g/L in aqueous solution. In yet another aspect of the solution does not contain any potassium hydroxide.

In an embodiment of this aspect, said salt of said phosphate compound comprises an ammonium salt or a metal salt, in particular an alkali metal salt, preferably a polyphosphate alkali metal salt, more preferably a potassium salt and a sodium salt, more preferably potassium polyphosphate, in particular potassium tripolyphosphate, selected from one or more of orthophosphate, pyrophosphate, cyclic phosphate, polyphosphate and superphosphate. In one embodiment, in said aqueous solution of disinfectant, the salt concentration of said phosphate compound is 2.5-2.8 wt %.

The aqueous disinfectant solution according to the present disclosure can be prepared according to methods known to those skilled in the art, such as simply mixing the components in a suitable container.

For example, a suitable amount of alkali metal or alkaline earth metal hydroxide (e.g. potassium hydroxide) and a salt of a phosphate compound (e.g. potassium tripolyphosphate) can be dissolved in a small amount of purified water, hypochlorite (e.g. sodium hypochlorite raw material) can be added to the above solution, and finally a suitable volume of purified water can be added.

According to another aspect of the present disclosure, the aqueous disinfectant solution according to the present disclosure can be used to disinfect medical devices. In one embodiment, said medical device is a hemodialysis machine.

### Examples

The aqueous disinfectant solutions of each embodiment were prepared by mixing the components in the amounts shown in Table 1 below.

Next, the storage stability and anti-microbial activity of the aqueous disinfectant solutions of each embodiment were tested according to the following experimental method. The results are also presented in Table 1.

### Experimental Method

### I. Bacterial Killing Test (Anti-Microbial Activity).

The disinfectant test is conducted in accordance with the Technical Specification 2002, Section 2.1.1 “Microbicide Test for Disinfectants” (see pages 15-28 of the specification).

### 1. Materials and Equipment

1) Test strain: Bacillus subtilis black variant (ATCC 9372) bacterium

2) Neutralizer: D/E Neutralizing broth

3) Diluent: tryptone saline solution (TPS)

4) Test carrier: sterile round stainless-steel sheet (diameter 12 mm, thickness 0.5 mm)

5) Culture medium: Tryptone soy agar medium (TSA), Tryptone soy broth medium (TSB)

### 2. Method

1) Testing basis: 2.1.1.5.6 and 2.1.1.7.5 (1) of “Technical Specification for Disinfection” (2002 edition)

2) Neutralizer identification test: The aqueous disinfectant solution prepared in each example was diluted with purified water at a ratio of 1:35 for 10.0 min, and the test temperature was 37° C. The test was repeated three times.

3) Killing test: The aqueous disinfectant solution prepared in each example was diluted with purified water at a ratio of 1:35, and the action time was 10.0 min, and the test temperature was 37° C. The test was repeated 3 times.

The logarithmic values of bacterial bactericide for aqueous solutions of disinfectants for each embodiment were calculated as follows.

Logarithm of kill=logarithm of the average concentration of viable bacteria in the control group−logarithm of the concentration of viable bacteria in the test group.

The effective chlorine content of the aqueous disinfectant solution of each example was measured before and after placement in accordance with the Technical Specification for Disinfectants 2002 “Determination of Effective Chlorine Content” (see section 2.2.1.2.1 on pages 110-111 of the specification) to calculate the rate of decline of effective chlorine and to determine its stability.

### II. Accelerated Stability Experiments

The aqueous disinfectant solutions of each example were packed in glass bottles and placed in a constant temperature oven at 54° C.±2° C. for 7 days. Before and after storage, the effective chlorine content of the aqueous disinfectant solution of each example was measured according to the Technical Specification for Disinfectants 2002 “Determination of Effective Chlorine Content” (see section 2.2.1.2.1 on pages 110-111 of the Specification) to calculate the rate of decrease of effective chlorine and to determine its stability.

TABLE 1

| | | | | | Hypochlorite in |
|---|---|---|---|---|---|
| | | | | | accelerated |
| | | | Effective | | experiments |
| | | | chlorine | Bacteria | The rate of |
| | | | (sodium | Germicidal | acidity decline |
| Example | KOH | KTPP | hypochlorite). | Logarithmic | (54° C., 7 |
| number | wt % | wt % | g/L | log | days) |
| 1 | 4.5 | 2.5 | 37.5 | 5.45 | 19.8% |
| 2 | 4.0 | 0.5 | 33 | 5.82 | 18.0% |
| 3 | 4.0 | 0.5 | 35 | 4.82 | 19.0% |
| 4 | 4.0 | 0.5 | 29.4 | 4.41 | 18.0% |
| 5 | 0 | 2.5 | 35 | 6.27 | 15.0% |
| 6 | 3.5 | 4 | 30 | 3.5 | 19.62% |
| 7 | 3.8 | 3 | 45 | 3.2 | 28.57% |
| 8 | 5 | 2 | 35 | 3.4 | 20.46% |
| 9 | 5 | 1.5 | 25 | 3.5 | 13.29% |
| 10 | 6 | 1 | 30 | 4.2 | 16.84% |
| 11 | 6 | 0.01 | 35 | 4.5 | 21.40% |
| 12 | 6 | 0.005 | 45 | 4.8 | 25.51% |
| 13 | 0 | 2.0 | 30 | 6.42 | 13.40% |
| 14 | 0 | 3.0 | 40 | 6.42 | 19.47% |
| 15 | 0 | 2.8 | 33 | 6.19 | 15.51% |

The results in Table 1 above show that the disinfectant aqueous solutions of each embodiment have a logarithm of killing bacterial spores of at least 3.2, which is higher than the requirement of >3 for medical disinfectants.

The decrease rate of hypochlorite in the accelerated experiment is less than 20%, which is equivalent to stability of at least 8 months.

Therefore, the aqueous disinfectant solution according to the present disclosure can meet the requirements of medical device disinfectants by having both higher stability and good microbicidal activity.

The samples 5 and 13 to 15 do not contain potassium hydroxide. These samples show a high stability and a log reduction greater than 6. They may also be useful as disinfectants, especially in cases where no fat and protein removal are required.

1-11. (canceled)

12. An aqueous disinfectant solution comprising:
20 g/L to 50 g/L of a hypochlorite;
3.5 wt % to 7.0 wt % of one or more metal hydroxides selected from an alkali metal hydroxide and an alkaline earth metal hydroxide; and
0.001 wt % 4.0 wt % of one or more salts of a phosphoric acid compound selected from orthophosphoric acid, pyrophosphoric acid, cyclic phosphoric acid, polyphosphoric acid, and superphosphoric acid.

13. The aqueous disinfectant solution according to claim 12, wherein the hypochlorite comprises sodium hypochlorite.

14. The aqueous disinfectant solution according to claim 12, wherein the hypochlorite comprises potassium hypochlorite.

15. The aqueous disinfectant solution according to claim 12, comprising 25 g/L to 45 g/L of the hypochlorite.

16. The aqueous disinfectant solution according to claim 12, comprising 28 g/L to 38 g/L of the hypochlorite.

17. The aqueous disinfectant solution according to claim 12, comprising 30 g/L to 35 g/L of the hypochlorite.

18. The aqueous disinfectant solution according to claim 12, wherein the metal hydroxide comprises sodium hydroxide or potassium hydroxide.

19. The aqueous disinfectant solution according to claim 12, wherein the alkali metal or alkaline earth metal hydroxide is present at a content of 4.0 wt % to 6.0 wt %.

20. The disinfectant aqueous solution according to claim 12, wherein the one or more salts of a phosphate compound comprises an ammonium salt or a metal salt.

21. The aqueous disinfectant solution according to claim 20, wherein the metal salt comprises a polyphosphate alkali metal salt.

22. The aqueous disinfectant solution according to claim 21, wherein the polyphosphate alkali metal salt comprises a potassium tripolyphosphate.

23. The aqueous disinfectant solution according to claim 12, wherein the one or more salts of a phosphate compound has a salt content of 0.01 wt % to 3.5 wt %.

24. The aqueous disinfectant solution according to claim 12, wherein the one or more salts of a phosphate compound has a salt content of 0.1 wt % to 1.0 wt %.

25. An aqueous disinfectant solution comprising:
30 g/L to 40 g/L of a hypochlorite; and
2.0 wt % to 3.0 wt % of one or more salts of a phosphoric acid compound selected from orthophosphoric acid, pyrophosphoric acid, cyclic phosphoric acid, polyphosphoric acid, and superphosphoric acid.

26. The aqueous disinfectant solution according to claim 25, wherein the hypochlorite comprises sodium hypochlorite or potassium hypochlorite.

27. The aqueous disinfectant solution according to claim 26, wherein the hypochlorite comprises sodium hypochlorite, and the sodium hypochlorite is present at a concentration of 33 g/L to 35 g/L.

28. The aqueous disinfectant solution according to claim 27, wherein the one or more salts of a phosphate compound comprises an ammonium salt or a metal salt.

29. The aqueous disinfectant solution according to claim 28, wherein the metal salt comprises a polyphosphate alkali metal salt.

30. The aqueous disinfectant solution according to claim 29, wherein the polyphosphate alkali metal salt comprises a potassium tripolyphosphate, and the sodium hypochlorite is present at a concentration of 2.5 wt % to 2.8 wt %.

31. A method comprising:
disinfecting a medical device with an aqueous disinfectant solution, the aqueous disinfectant solution comprising
20 g/L to 50 g/L of a hypochlorite;
3.5 wt % to 7.0 wt % of one or more metal hydroxides selected from an alkali metal hydroxide and an alkaline earth metal hydroxide; and
0.001 wt % 4.0 wt % of one or more salts of a phosphoric acid compound selected from orthophosphoric acid, pyrophosphoric acid, cyclic phosphoric acid, polyphosphoric acid, and superphosphoric acid.
